(19) United States
(12) Patent Application Publication
(10) Pub. No.: US 2011/0151000 A1
(43) Pub. Date: Jun. 23, 2011

(54) NITRIC OXIDE-RELEASING COMPOSITIONS, DEVICES AND METHODS

(76) Inventors: William J. Schultz, North Oaks, MN (US); Louis C. Haddad, Mendota Heights, MN (US); John C. Stendahl, St. Paul, MN (US); Corey J. Radloff, St. Paul, MN (US); Stephanie F. Bernatchez, Woodbury, MN (US); Matthew T. Scholz, Woodbury, MN (US)

(21) Appl. No.: 12/988,700

(22) PCT Filed: Apr. 20, 2009
(86) PCT No.: PCT/US2009/041090

§ 371 Date: Mar. 8, 2011

Related U.S. Application Data
(60) Provisional application No. 61/046,659, filed on Apr. 21, 2008.

(51) Int. Cl. A61K 9/14; A61K 9/00; A61K 33/00; B05D 3/00; A61P 9/10; A61P 17/02; A61P 9/12; B65D 85/84

(52) U.S. Cl. 424/484; 424/490; 424/718; 427/2.14; 206/524.1

(57) ABSTRACT

Nitric oxide releasing compositions, which comprise nanoparticles having an exterior surface comprising solid amorphous silica, the exterior surface having nitrosothiol-containing groups attached thereto, the nanoparticles being dispersible in an aqueous system, devices including the compositions, and methods of making and using the compositions and devices are disclosed.

### CROSS REFERENCE TO RELATED APPLICATIONS

This application claims the benefit of U.S. Provisional Application No. 61/046,659, filed Apr. 21, 2008, which is incorporated herein by reference in its entirety.

### BACKGROUND

Nitric oxide (NO) is a short-lived, gaseous free radical signaling molecule that regulates many important physiological processes, including circulation, angiogenesis, wound healing, immune responses, neurotransmission, cell signaling, cell proliferation, and cell survival. NO deficiencies are associated with multiple pathological conditions, including hypertension, atherosclerosis, and chronic wounds.

NO is generated in vivo by the action of nitric oxide synthases on L-arginine. Because NO degrades quickly in vivo (t_{1/2}<5 sec), endogenous S-nitrosothiols serve an important role in the body to regulate NO concentrations. These nitrosothiols, which can release NO, are formed when NO is bound in vivo to thiolated molecules such as serum albumin, cysteine and glutathione.

Certain exogenous NO donor materials, which can chemically store and release NO, have been proposed for therapeutic use. Such materials include NO donating groups such as nitrosothiols, diazeniumdiolates, nitrosamines, NO-metal complexes, and organic nitrites and nitrates. N-diazeniumdiolate NO donor materials have been given considerable attention because they are stable under ambient conditions, and can decompose in aqueous media to generate NO. However, the synthesis of these materials can be difficult (requiring reactions with pressurized nitric oxide gas) and not easily scaled up, and there has been some concern that if some of the diazeniumdiolate and/or its decomposition products leached into the blood, a toxicity issue could potentially arise.

Exogenous S-nitrosothiols have also been given some attention, particularly because of their similarity to the endogenous S-nitrosothiols. However, the usefulness of these materials has been limited by the relative instability of the S—NO bond. For example, small molecule nitrosothiols have been reported to have half lives in aqueous solution on the order of only minutes to days.

Accordingly, there continues to be an interest in and a need for new NO donor materials and compositions.

### SUMMARY

It has now been found that nitrosothiol groups attached to a solid amorphous silica surface of silica nanoparticles have improved stability and can deliver NO. The silica nanoparticles are dispersible in an aqueous system, and for certain embodiment, the nanoparticles are distributed in a hydrogel monomer or a hydrogel. Such compositions can be used to release NO to a surface or to an adjacent mammalian tissue to provide a therapeutic effect, such as to promote healing in certain types of wounds, provide an antimicrobial effect, inhibit platelet adhesion and blood coagulation, stimulate angiogenesis, or to enhance blood flow to increase absorption of a drug.

In one embodiment, there is provided a composition for releasing nitric oxide, the composition comprising nanoparticles having an exterior surface comprising solid amorphous silica, wherein nitrosothiol-containing groups are attached to the surface, and wherein the nanoparticles are dispersible in an aqueous system.

In another embodiment, there is provided a medical device comprising a surface and a composition for releasing nitric oxide adjacent the surface, the composition comprising nanoparticles having an exterior surface comprising solid amorphous silica, wherein nitrosothiol-containing groups are attached to the surface, and wherein the nanoparticles are dispersible in an aqueous system.

In another embodiment, there is provided a product comprising the above medical device and a package impervious to water, water vapor, ultraviolet light, and visible light, wherein the composition for releasing nitric oxide is enveloped by the package.

In another embodiment, there is provided a method of treating a subject with nitric oxide, the method comprising:

providing a composition for releasing nitric oxide, the composition comprising nanoparticles having an exterior surface comprising solid amorphous silica, wherein nitrosothiol-containing groups are attached to the surface, and wherein the nanoparticles are dispersible in an aqueous system;

contacting the subject with the composition; and

releasing nitric oxide at a location where the composition or the medical device contacts the subject.

In another embodiment, there is provided a method of treating a subject with nitric oxide, the method comprising:

providing a medical device comprising a surface and a composition for releasing nitric oxide adjacent the surface, the composition comprising nanoparticles having an exterior surface comprising solid amorphous silica, wherein nitrosothiol-containing groups are attached to the surface, and wherein the nanoparticles are dispersible in an aqueous system;

contacting the subject with the medical device; and

releasing nitric oxide at a location where the medical device contacts the subject.

In another embodiment, there is provided a kit comprising a composition for releasing nitric oxide; the composition comprising nanoparticles having an exterior surface comprising solid amorphous silica, wherein nitrosothiol-containing groups are attached to the surface, and wherein the nanoparticles are dispersible in an aqueous system; and an activating agent.

In another embodiment, there is provided method of making a composition for releasing nitric oxide, the method comprising:

providing solid amorphous silica nanoparticles;

bonding thiol-containing groups to an exterior surface of the solid amorphous silica nanoparticles;

bonding stabilizing groups comprising hydrophilic groups to the exterior surface of the solid amorphous silica nanoparticles; and

nitrosylating the thiol-containing groups to form nitrosothiol-containing groups.

In another embodiment, there is provided a composition for releasing nitric oxide, the composition comprising:

a first part comprising nanoparticles having an exterior surface comprising solid amorphous silica, wherein thiol-containing groups are attached to the surface, and wherein the nanoparticles are dispersible in an aqueous system; and

a second part comprising a nitrite source.

In another embodiment, there is provided a medical device comprising a surface and a composition for releasing nitric oxide adjacent the surface; the composition comprising:

a first part comprising nanoparticles having an exterior surface comprising solid amorphous silica, wherein thiol-containing groups are attached to the surface, and wherein the nanoparticles are dispersible in an aqueous system; and

a second part comprising a nitrite source.

In another embodiment, there is provided a method of treating a subject with nitric oxide, the method comprising:

providing a composition for releasing nitric oxide, the composition comprising: a first part comprising nanoparticles having an exterior surface comprising solid amorphous silica, wherein thiol-containing groups are attached to the surface, and wherein the nanoparticles are dispersible in an aqueous system; and a second part comprising a nitrite source;

combining the first part of the composition with the second part of the composition to provide a composition, wherein nitrosothiol-containing groups are attached to the surface of the nanoparticles;

contacting the subject with the composition for releasing nitric oxide; and

releasing nitric oxide at a location where the composition contacts the subject.

In another embodiment, there is provided a method of treating a subject with nitric oxide, the method comprising:

providing a medical device comprising a surface and a composition for releasing nitric oxide adjacent the surface; the composition comprising: a first part comprising nanoparticles having an exterior surface comprising solid amorphous silica, wherein thiol-containing groups are attached to the surface, and wherein the nanoparticles are dispersible in an aqueous system; and a second part comprising a nitrite source;

combining the first part of the composition with the second part of the composition to provide a composition, wherein nitrosothiol-containing groups are attached to the surface of the nanoparticles;

contacting the subject with the medical device; and

releasing nitric oxide at a location where the medical device contacts the subject.

The above summary of the present invention is not intended to describe each disclosed embodiment or every implementation of the present invention. The description that follows more particularly exemplifies illustrative embodiments. In several places throughout the description, guidance is provided through lists of examples, which examples can be used in various combinations. In each instance, the recited list serves only as a representative group and should not be interpreted as an exclusive list.

### DEFINITIONS

As used herein, the term “solid amorphous silica” refers to a fully densified silica, which is typically made by condensing a silicate, for example, sodium silicate, tetraethyl orthosilicate, or tetramethyl orthosilicate, under basic conditions, for example, at a pH of about 8 to about 10. This is distinguished from a co-condensed silica, for example, co-condensation of tetraethyl orthosilicate with a di- or tri-aminoalkoxysilane, which is porous and not fully densified. However, minor amounts, for example, not more than 5, 1 or 0.5 percent by weight, of an organic group may be present in the solid amorphous silica.

As used herein, the term “solid amorphous silica nanoparticles” refers to essentially spherically shaped, fully densified silica particles having a diameter of about 1 nanometer to about 100 nanometers. These nanoparticles remain substantially non-aggregated. For certain embodiments, the nanoparticles are relatively uniform in size. Alternatively, for certain embodiments, the nanoparticles are a mixture of two or more different sizes and are polydispersed.

As used herein, the term “dispersible in an aqueous system” indicates that the material which is dispersible, for example, the nanoparticles, can be uniformly and stably suspended in an aqueous system. Stably suspended means that the dispersion remains uniform with no apparent settling when held at 23° C. for at least one week. Such mixtures are transparent or translucent and remain so with the nanoparticles suspended for weeks, preferably months, or even longer.

As used herein, the term “aqueous system” refers to a combination of water and at least one water dispersible compound.

As used herein, the term “water dispersible compound” refers to a compound which can be uniformly and stably suspended or dissolved in water.

As used herein, the term “hydrogel” refers to a hydrophilic polymer that absorbs water, but which is insoluble in water because of the presence of a three-dimensional network formed from crosslinks. The crosslinks may be covalent or ionic. The crosslinks may alternatively or additionally be hydrogen bonds and/or polymer chain entanglements.

The hydrogel behaves as a solid or semisolid in that it will not flow under ambient conditions (23° C. and atmospheric pressure). The absorbed water can include bound water and free water. Bound water is associated with hydrophilic groups located along the polymer chain. Additional water that is absorbed by the hydrogel is free water which fills the voids and pores of the hydrogel. For certain embodiments, useful hydrogels absorb at least 40% by weight based on the hydrogel's weight in an anhydrous state. The hydrogels are typically transparent or translucent, regardless of their degree of hydration. Hydrogels are generally distinguishable from hydrocolloids, which typically comprise a hydrophobic matrix that contains dispersed hydrophilic particles.

As used herein, the term “substantially dehydrated” refers to a hydrogel which contains at most about 10 percent by weight of water, preferably at most about 5 percent by weight of water.

As used herein, the term “transparent” refers to a characteristic of the composition which allows a person to sufficiently see through the composition, such that a condition of a tissue or other structure immediately behind the composition can be observed. Examples of conditions that may be observed include a degree of redness of the tissue, a degree of closure of a wound, and an amount of wound exudate. The term “transparent” refers to the appearance to the naked eye when viewed through a 0.5 cm, preferably a 1 cm length path cell, and includes clear and translucent, although clear is preferred.

As used herein, the term “fluid” refers to a material which is a liquid in the temperature range of room temperature to body temperature.

As used herein, the term “aqueous composition” refers to a liquid composition comprised of at least 50 percent by weight of water.

The term “amphiphilic compound” refers to a compound which has both a hydrophilic region and a hydrophobic region.

The terms “comprises”, “comprising” and variations thereof do not have a limiting meaning where these terms appear in the description and claims.

Unless a particular numerical range recited herein requires that the numbers therein be only integers, the recitation herein of numerical ranges by endpoints is intended to include all numbers subsumed within that range (e.g. 1 to 5 includes 1, 1.5, 2, 2.75, 3, 3.80, 4, and 5).

As used herein, “a” or “an” means “at least one” or “one or more” unless otherwise indicated. In addition, the singular forms “a”, “an”, and “the” include plural referents unless the content clearly dictates otherwise. Thus, for example, reference to a composition containing “a compound” includes a mixture of two or more compounds. As used in this specification and the appended claims, the term “or” is generally employed in its sense including “and/or” unless the content clearly dictates otherwise.

Unless otherwise indicated, all numbers expressing quantities of ingredients, measurement of properties and so forth used in the specification and claims are to be understood as being modified in all instances by the term “about.” Accordingly, unless indicated to the contrary, the numerical parameters set forth in the present specification and claims are approximations that can vary depending upon the desired properties sought to be obtained by those skilled in the art utilizing the teachings of the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviations found in their respective testing measurements.

### DETAILED DESCRIPTION OF ILLUSTRATIVE EMBODIMENTS OF THE INVENTION

The present invention provides nanoparticles with a solid amorphous silica surface to which nitrosothiol groups are attached. The silica nanoparticles are dispersible in an aqueous system and can be dispersed in a hydrogel for use as a wound dressing or for other therapeutic applications. The nanoparticles can release nitric oxide (NO). Moreover, the rate of NO release has been found to be more controlled than that of small molecule nitrosothiols. In certain embodiments, the nitrosothiol groups on the nanoparticles have a half-life on the order of days or weeks in an aqueous system, and can have a considerably longer half-life in a dehydrated composition. By comparison, small molecule nitrosothiols are known to have half-lives on the order of minutes to days. Some examples of nitrosothiol lifetimes for various molecules are given in W. R. Mathews, S. W. Kerr, The Journal of Pharmacology and Experimental Therapeutics, 267, 1529 (1993).

In one embodiment, there is provided a composition for releasing nitric oxide, the composition comprising nanoparticles having an exterior surface comprising solid amorphous silica, wherein nitrosothiol-containing groups are attached to the surface, and wherein the nanoparticles are dispersible in an aqueous system.

Two-part compositions for releasing nitric oxide may be desirable under certain circumstances, for example, where storage conditions are severe or uncontrolled. Accordingly, in another embodiment, there is provided a composition for releasing nitric oxide, the composition comprising: a first part comprising nanoparticles having an exterior surface comprising solid amorphous silica, wherein thiol-containing groups are attached to the surface, and wherein the nanoparticles are dispersible in an aqueous system; and a second part comprising a nitrite source.

A fluid can be used to facilitate combining the second part of the composition with the first part of the composition. For example, a fluid can provide flow of the second part to the first part as well as mixing of the second part with the first part or diffusion of the second part into the first part. For certain of these embodiments, the fluid is an aqueous composition. For certain embodiments, the nitrite source is in an aqueous composition. Suitable aqueous compositions contain at least 50 percent by weight of water, preferably at least 75 or at least 90 percent by weight of water.

For certain embodiments, including any one of the above embodiments of two-part compositions, the nitrite source is a nitrite salt. Suitable nitrite salts include, for example, sodium, potassium, and ammonium salts. Ammonium salts include NR₄⁺ where R is hydrogen, C₁₋₄ alkyl, or a combination there. In an alternative embodiment, nitrous acid or a combination of nitrous acid and a nitrite salt is used for nitrosylating the thiol-containing groups.

For certain embodiments, including any one of the above embodiments of two-part compositions, the aqueous system has an acidic pH. The acidic pH provides favorable conditions for the nitrosylation, which consumes H⁺ in the formation of the nitrosothiol groups. For certain embodiments, the pH is preferably less than or equal to about 6 or about 5. For certain embodiments, the pH is at least about 2 or about 3.

For certain embodiments, including any one of the above compositions, the nanoparticles are solid amorphous silica nanoparticles. The solid amorphous silica nanoparticles are substantially spherical in shape, have a diameter of about 1 nanometer to about 100 nanometers, and, except for groups attached to the surface, are composed essentially of fully densified amorphous silica. These nanoparticles are relatively uniform in size, or a mixture of sizes can be used. These nanoparticles remain substantially non-aggregated, for example, as a colloidal dispersion. Aggregation is undesirable, because it can result in precipitation, gelation, or a substantial increase in viscosity.

Silica nanoparticles, which can be used as starting materials in preparing the present compositions, can be provided as a colloidal dispersion of the nanoparticles in a liquid media. Such sols can be hydrosols where water is the liquid media or mixed sols where the liquid media comprises both water and an organic liquid. See, for example, the descriptions of the techniques and forms given in U.S. Pat. No. 2,801,185 (Iler) and U.S. Pat. No. 4,522,968 (Das et al.) as well as those given by R. K. Iler in The Chemistry of Silica, John Wiley & Sons, New York (1979). Useful silica hydrosols are available in a variety of particle sizes and concentrations from, e.g., Nyacol Products, Inc. in Ashland, Md.; Nalco Chemical Company in Oakbrook, Ill.; and E. I. dupont de Nemours and Company in Wilmington, Del. Concentrations of from about 10 to about 50 percent by weight of silica nanoparticles in water are generally useful. If desired, silica hydrosols can be prepared, for example, by partially neutralizing an aqueous solution of an alkali metal silicate with acid to a pH of about 8 to about 9 (such that the resulting sodium content of the solution is less than about 1 percent by weight based on sodium oxide). Other methods of preparing silica hydrosols, such as electrodialysis, ion exchange of sodium silicate, hydrolysis of silicon compounds, and dissolution of elemental silicon, are described by Iler, supra.

For certain embodiments, including any one of the above embodiments which includes solid amorphous silica nanoparticles, the solid amorphous silica nanoparticles have a density of about 2.0 to about 2.3 grams/cm³. For certain of these embodiments, the density of the solid amorphous silica nanoparticles is about 2.1 to about 2.2. For certain of these embodiments, the density of the solid amorphous silica nanoparticles is about 2.2.

Alternatively, for certain embodiments, including any one of the above embodiments which includes nanoparticles having an exterior surface comprising solid amorphous silica, rather than being solid amorphous silica nanoparticles, the nanoparticles have an exterior surface comprising solid amorphous silica which surrounds a core, and wherein the core comprises a metal oxide selected from the group consisting of zirconium oxide, titanium oxide, aluminum oxide, zinc oxide, and cerium oxide. Such nanoparticles can be made by starting with colloidal particles of the metal oxide and depositing solid amorphous silica on the surface of the colloidal particles. This can be done by condensing a silicate, as described above, on the surface of the colloidal particles. Methods for depositing a silica layer on a particle are known and described, for example, in Philipse, et al., Langmuir, 10, 4451-4458 (1994).

As indicated above, nitrosothiol groups include the —S—NO functional group. These functional groups can be covalently attached to the surface of the nanoparticles via a tether group, for example, by Si—O—Si bonds. For example, Si—OH groups on the surface of the nanoparticles can be condensed with a Si—OH group bonded to a tether group to which a —S—NO group is bonded. For certain embodiments, a compound of the formula:

H—S—Y—Si(OR¹)₃

is used for this purpose, wherein R¹ is C₁₋₃ alkyl, and Y is a tether group having a chain length of 2 to 10 atoms, and wherein the atoms in the chain can be carbon, oxygen, and nitrogen, at least two of the atoms in the chain being adjacent carbon atoms, and any two oxygen atoms, nitrogen atoms, or oxygen and nitrogen atoms being separated by at least two carbon atoms. For certain embodiments, Y is C₂₋₆ alkylene. The alkoxysilyl —Si(OR¹)₃ groups can be hydrolyzed to —Si(OH)₃, which can then condense with Si—OH groups on the surface of the nanoparticles to provide thiol groups attached to the surface of the nanoparticles. The thiol groups can be reacted with a source of nitrite (NO₂⁻) ions to provide nitrosthiol groups. As indicated above, for embodiments where the composition is a two-part composition, the nitrite source is in the second part.

For certain embodiments, a compound of the formula:

Z—Y—Si(OR¹)₃

is used for this purpose, wherein Z is isocyanato, glycidyl, haloalkyl, or an acid anhydride group, R¹ is C₁₋₃ alkyl, and Y is a tether group having a chain length of 2 to 10 atoms, and wherein the atoms in the chain can be carbon, oxygen, and nitrogen, at least two of the atoms in the chain being adjacent carbon atoms, and any two oxygen atoms, nitrogen atoms, or oxygen and nitrogen atoms being separated by at least two carbon atoms. For certain embodiments, Y is C₂₋₆ alkylene. The alkoxysilyl —Si(OR¹)₃ groups can be hydrolyzed to —Si(OH)₃, which can then condense with Si—OH groups on the surface of the nanoparticles to provide isocyanato, glycidyl, haloalkyl, or an acid anhydride groups attached to the surface of the nanoparticles. These groups can be reacted with amine groups similar to those found on sulfhydryl containing amino acids or hydroxyl groups similar to those found on sulfhydryl containing amino acids, peptides, glycerols, and carbohydrates. For certain embodiments, the isocyanato-, glycidyl-, haloalkyl-, or acid anhydride-silane of the above formula, preferably the isocyanato-silane, may be reacted with the sulfhydryl containing molecule (with amino or hydroxyl groups) in solution and then reacted to attach to the surface of the particle.

In addition to —Si(OR¹)₃ groups, other coupling groups may be used such as dialkoxysilyl, mono-, di, and trichlorosilyl, and the like, depending upon which other groups are present in the molecule. For example, a chlorosilyl group may not be stable with the sulfhydryl group present, but would be with an isocyanato, epoxy, haloalkyl, or anhydride group.

For certain embodiments, including any one of the above embodiments, preferably the nanoparticles further comprise stabilizing groups attached to the surface of the nanoparticles, and wherein the stabilizing groups comprise hydrophilic groups. Such hydrophilic groups, if not attached to the nanoparticles, would be water soluble. Suitable hydrophilic groups are selected from the group consisting of —OCH₂CH₂—, —COOH, a salt of —COOH, —CH(OH)CH₂OH, —CH(OH)CH(OH)—, —SO₃H, a salt of —SO₃H, a quaternary amino group, a monosaccharide group, an oligosaccharide group, a plurality of any of the preceding groups, and a combination thereof. A plurality of any of these groups refers to oligomeric and polymeric groups where the repeating units include these groups. The stabilizing groups should stabilize the nanoparticles by preventing agglomeration of the particles and keeping the nanoparticles suspended in the aqueous system. The stabilizing groups can also stabilize the release of NO to provide a more sustained release of NO as compared with nanoparticles which do not have such stabilizing groups at the surface of the nanoparticles. The manner in which the release of NO is stabilized is not known. However, among other possibilities, the stabilizing groups may cause steric inhibition of reactions between adjacent or inter-particle nitrosothiol groups that would otherwise form —S—S— bonds and cause undesired or premature release of NO. The stabilizing groups may also provide a barrier to protect the nitrosothiol groups from contacting or reduce the rate of nitrosothiol groups contacting other molecules that could trigger release of NO.

The above described hydrophilic groups which are monovalent are associated with the above described stabilizing group chains as a terminal group, as at least one group attached along the chain, or as a combination of at least one group attached along the chain and a terminal group. The above described hydrophilic groups which are divalent are associated with the above described stabilizing group chain as part of the chain, as a repeating unit in the chain, and in certain embodiments, preferably as a terminal part of the chain. When a divalent group is the end group of a terminal part of the chain, one of the valencies is attached to a hydrogen or C₁₋₄ alkyl group, preferably a hydrogen, methyl, or ethyl group. Terminal groups are attached to the terminal part of the chain, which is furthest from the nanoparticle surface.

The stabilizing groups have a sufficient chain length to stabilize the nitrosothiol groups from releasing NO. However, the chain of a stabilizing group may have branches anywhere along this chain. For certain embodiments, including any one of the above embodiments which includes stabilizing groups, the stabilizing groups have a chain length of at least about 10 in-chain atoms, wherein in-chain atoms of the hydrophilic groups, other than hydrogen, are included.

For certain embodiments, including any one of the above embodiments which includes stabilizing groups, the stabilizing groups have a chain length of not more than about 200 in-chain atoms, wherein in-chain atoms of the hydrophilic groups, other than hydrogen, are included. For certain of these embodiments, the stabilizing groups have a chain length of not more than about 150 in-chain atoms.

For certain embodiments, including any one of the above embodiments which includes stabilizing groups, the stabilizing groups have a chain length of not more than about 100 in-chain atoms, wherein in-chain atoms of the hydrophilic groups, other than hydrogen, are included.

For certain embodiments, including any one of the above embodiments which includes stabilizing groups, the stabilizing groups have a chain length of about 20 to about 150 in-chain atoms, wherein in-chain atoms of the hydrophilic groups, other than hydrogen, are included.

For certain embodiments, including any one of the above embodiments which includes stabilizing groups, the stabilizing groups have a chain length of about 20 to about 75 in-chain atoms, wherein in-chain atoms of the hydrophilic groups, other than hydrogen, are included.

For certain embodiments, including any one of the above embodiments which includes stabilizing groups, the stabilizing groups comprise a chain wherein the atoms of the chain are selected from the group consisting of carbon, oxygen, nitrogen, sulfur, and silicon. For example, the chain can include at least one of C₂₋₁₄ alkylene, and optionally at least one of —O—, —S—, —SO₂—, —SO₂—NR²—, —NR²—SO₂—, —NR²—SO₂—NR²—, —NR2-C(O)—, —C(O)—NR2-, —NH—C(O)—NH—, —O—C(O)—NH—, —O—C(O)—, —C(O)—O—, —C(O)—, —CH(OH)—, or a combination thereof, wherein R² is H or methyl. For certain of these embodiments, the atoms of the chain are carbon, oxygen, and nitrogen. For certain of these embodiments, the atoms of the chain are carbon and oxygen. For certain embodiments, any of these embodiments further includes a silicon atom, preferably one silicon atom.

For certain embodiments, including any one of the above embodiments which includes stabilizing groups, the stabilizing groups comprise poly(ethylene glycol) chains. Poly(ethylene glycol) chains refers to oligomeric and polymeric groups of the formula (—OCH₂CH₂)ₘO— wherein m is at least 2. For certain embodiments, m is not more than 100, not more than 50, or not more than 25.

Stabilizing groups can be attached to the surface of the nanoparticles by Si—O—Si bonds. For example, Si—OH groups on the surface of the nanoparticles can be condensed with Si—OH groups bonded to molecules which can be used as stabilizing groups when attached to the nanoparticles. For certain embodiments, a compound of the formula:

R—(OCH₂CH₂)ₓ—(OCH₂CH(CH₃))_{y}-A-X—Si(OR¹)₃

is used for this purpose, wherein R is hydrogen or C₁₋₄ alkyl; x is an integer from 10 to 100; y is an integer from 2 to 20; X is C₂₋₅ alkylene; A is —O—, —NR′—, —NH—C(O)—NH—, —O—C(O)—NH—, —OC(O)—, —NH—C(O)—, —NH—CH₂—CH(OH)—, or —O—CH₂CH(OH)—; R′ is hydrogen or C₁₋₄ alkyl; and R¹ is C₁₋₃ alkyl. For certain embodiments, R is methyl, ethyl, or propyl, x is an integer from 15 to 50, y is an integer from 2 to 15, X is —CH₂CH₂CH₂—, A is —NH—C(O)—NH—, and R¹ is methyl or ethyl.

For certain embodiments, a compound of the formula:

R—(OCH₂CH₂)ₙO—X—Si(OR¹)₃

is used for this purpose, wherein R is hydrogen or C₁₋₄ alkyl, n is an integer from 5 to 20, X is C₂₋₅ alkylene, and R¹ is C₁₋₃ alkyl. For certain embodiments, R is methyl, ethyl, or propyl, n is an integer from 7 to 15, X is —CH₂CH₂CH₂—, and R¹ is methyl or ethyl.

The alkoxysilyl —Si(OR¹)₃ groups of the compounds of the above formulas can be hydrolyzed to —Si(OH)₃, which can then condense with Si—OH groups on the surface of the nanoparticles to provide stabilizing groups attached to the surface of the nanoparticles.

For certain embodiments, a compound of the formula:

W—X—Si(OR¹)₃

is used for this purpose, wherein W is a monosaccharide or an oligosaccharide, and includes a connecting group which covalently attaches the saccharide to X; X is C₂₋₅ alkylene, and R¹ is C₁₋₃ alkyl. Suitable connecting groups include, for example, —C(O)N—, —C(O)O—, and the like. For certain embodiments, the oligosaccharide contains up to six saccharide units. For certain embodiments, X is —CH₂CH₂CH₂—, and R¹ is methyl or ethyl. The alkoxysilyl —Si(OR¹)₃ groups can be hydrolyzed to —Si(OH)₃, which can then condense with Si—OH groups on the surface of the nanoparticles to provide stabilizing groups attached to the surface of the nanoparticles.

For certain embodiments, including any one of the above embodiments, the nanoparticles, whether nanoparticles having an exterior surface comprising solid amorphous silica or solid amorphous silica nanoparticles, are dispersed in an aqueous system. The aqueous system can be a combination of water and at least one water dispersible compound. Such water dispersible compounds include hydrophilic groups. For certain embodiments, the water dispersible compounds are liquid or low-melting (<70° C., preferably <40° C.) solid organic compounds with hydrophilic groups. Such hydrophilic groups include, for example, one or more of —OH, —C(O)OH, —C(O)—NR³—, —CH₂CH₂O—, and combinations thereof, wherein R³ is H, methyl, or vinyl. For certain of these embodiments, the water dispersible compound is selected from the group consisting of a lower alcohol, propylene glycol, glycerol, poly(ethylene glycol), a hydrogel monomer, an amphiphilic compound, and a combination thereof. For certain embodiments, the water dispersible compound is a lower alcohol. Lower alcohols include, for example, methanol, ethanol, isopropanol, and n-propanol. Alternatively, for certain embodiments, the water dispersible compound is a hydrogel monomer.

Suitable hydrogel monomers can be ethylenically unsaturated. Examples of those with one ethylenically unsaturated group per monomer molecule include, for example, 2-hydroxyethyl(meth)acrylate, 2-hydroxypropyl(meth)acrylate, 3-hydroxypropyl(meth)acrylate, 4-hydroxybutyl(meth)acrylate, caprolactone(meth)acrylate, (meth)acrylic acid, β-carboxyethyl(meth)acrylate, tetrahydrofurfuryl(meth)acrylate, (meth)acrylonitrile, (meth)acrylamide, N-(2-hydroxyethyl)(meth)acrylamide, N-methyl(meth)acrylamide, N,N-dimethyl(meth)acrylamide, diacetone acrylamide, N-vinyl-2-pyrrolidone, N-vinylcaprolactam, poly(alkylene oxide (meth)acrylate (e.g., poly(ethylene glycol) methyl ether(meth)acrylate, poly(ethylene glycol)(meth)acrylate, poly(propylene glycol)(meth)acrylate, and poly(ethylene oxide-co-propylene oxide(meth)acrylate)).

Suitable hydrogel monomers with two ethylenically unsaturated groups per monomer molecule include, for example, alkoxylated di(meth)acrylates. Examples of alkoxylated di(meth)acrylates include, but are not limited to, poly(alkylene oxide)di(meth)acrylates such as poly(ethylene glycol)di(meth)acrylates, poly(propylene glycol)di(meth)acrylates, and poly(ethylene glycol-ran-propylene glycol)di(meth)acrylate; alkoxylated diol di(meth)acrylates such as ethoxylated butanediol di(meth)acrylates, propoxylated butanediol di(meth)acrylates, and ethoxylated hexanediol di(meth)acrylates; alkoxylated trimethylolpropane di(meth)acrylates such as ethoxylated trimethylolpropane di(meth)acrylate and propoxylated trimethylolpropane di(meth)acrylate; and alkoxylated pentaerythritol di(meth)acrylates such as ethoxylated pentaerythritol di(meth)acrylate and propoxylated pentaerythritol di(meth)acrylate.

Suitable hydrogel monomers with three ethylenically unsaturated groups per monomer molecule include, for example, alkoxylated tri(meth)acrylates. Examples of alkoxylated tri(meth)acrylates include, but are not limited to, alkoxylated trimethylolpropane tri(meth)acrylates such as ethoxylated trimethylolpropane tri(meth)acrylates, propoxylated trimethylolpropane tri(meth)acrylates, and ethylene oxide/propylene oxide copolymer trimethylolpropane tri(meth)acrylates; alkoxylated pentaerythritol tri(meth)acrylates such as ethoxylated pentaerythritol tri(meth)acrylates, and alkoxylated glycerol tri(meth)acrylates such as ethoxylated glycerol tri(meth)acrylates.

Suitable hydrogel monomers with at least four ethylenically unsaturated groups per monomer include, for example, alkoxylated tetra(meth)acrylates and alkoxylated penta(meth)acrylates. Examples of alkoxylated tetra(meth)acrylates include alkoxylated pentaerythritol tetra(meth)acrylates such as ethoxylated pentaerythritol tetra(meth)acrylates.

For certain embodiments, in order for the ultimate hydrogel to be sufficiently crosslinked, preferably, the average number of ethylenically unsaturated groups (e.g., (meth)acryloyl groups) per hydrogel monomer molecule is equal to at least 1.2. This is accomplished by including a sufficient amount of hydrogel monomer having two or more ethylenically unsaturated groups. For example, the hydrogel monomers can contain at least one (meth)acrylate having two (meth)acryloyl groups per monomer molecule or can contain a mixture of at least one (meth)acrylate having two (meth)acryloyl groups per monomer molecule in combination with at least one (meth)acrylate having one (meth)acryloyl group per monomer molecule. In another example, the hydrogel monomers can contain at least one (meth)acrylate having three (meth)acryloyl groups per monomer molecule or can contain a mixture of at least one (meth)acrylate having three (meth)acryloyl groups per monomer molecule in combination with at least one (meth)acrylate having one (meth)acryloyl group per monomer molecule, two (meth)acryloyl groups per monomer molecule, or a mixture thereof.

Suitable hydrogel monomers can also include hydrophilic polyols or polyamines for use in preparing polyurethane and polyurea hydrogels. Examples of hydrophilic polyols include, for example, poly(ethylene glycols), a polyether triol of a copolymer of ethylene oxide and propylene oxide, a glycerin initiated polyoxyethylene glycol triol, and the like. Polyamines include, for example, amine terminated analogs of these polyols, available, for example, as JEFFAMINES (Huntsman Petrochemical Corp., Salt Lake City, Utah).

Suitable amphiphilic compounds may also be included, for example, nonionic surfactants such as poly(ethylene glycol)monolaurate, poly(ethylene glycol)monooleate, poly(ethylene glycol)myristyl tallow ether, poly(ethylene glycol)methyl ether-block-poly(ε-caprolactone), poly(ethylene glycol)-block-polypropylene glycol)-block-poly(ethylene glycol), and the like may be used.

For certain embodiments, including any one of the above embodiments other than those where the nanoparticles, whether nanoparticles having an external surface comprising solid amorphous silica or solid amorphous silica nanoparticles, are dispersed in an aqueous system, the composition further comprises a hydrogel; wherein the nanoparticles are distributed in the hydrogel. Suitable hydrogels include natural polymers covalently or ionically crosslinked to form hydrogels. Suitable natural polymers for this purpose include polysaccharides such as agar, guar gum, xanthan gum, alginic acid and alginates, chitin, chitosan, cellulose and cellulose derivatives such as hydroxyethyl cellulose, hydroxypropylmethyl cellulose, carboxymethylcellulose, and the like, and pectin. Suitable hydrogels also include conventional synthetic hydrogels (e.g., a polymer made from at least one of the above described hydrogel monomers), silicone hydrogels (e.g., a copolymer of 2-hydroxyethyl methacrylate and methacryloyloxyethyltris(trimethoxysilyloxy)silane or the like), polyurethane hydrogels (e.g., a hydrophilic polyol reacted with a di- or polyisocyanate), and polyurea hydrogels (e.g., a hydrophilic amine terminated polyethylene glycol or polyethylene glycol/polypropylene glycol copolymer reacted with a di- or polyisocyanato functional compound). Many hydrogels are known and described, for example, in Encyclopedia of Polymer Science and Technology, John Wiley & Sons Inc., Vol 2, pages 691-722 (2002) and International Publication No. WO 2007/146722. For certain of these embodiments, the hydrogel comprises the polymerization product of at least one ethylenically unsaturated compound containing hydrophilic groups. For certain of these embodiments, the at least one ethylenically unsaturated compound includes polyethylene glycol acrylate methyl ether, 2-hydroxyethyl acrylate, 2-hydroxyethyl methacrylate, and ethylene oxide-propylene oxide copolymer dimethacrylate (i.e., poly(ethylene oxide-ran-propylene oxide) dimethacrylate). For certain of these embodiments, the hydrogel comprises a crosslinked poly(N-vinyl lactam), such as poly(N-vinyl pyrrolidone). Hydrogels of this nature are further described in Applicant's pending application, U.S. Ser. No. 61/022,036. For certain other embodiments, the hydrogel comprises the polymerization (e.g., photopolymerization) product of ethoxylated trimethylolpropane triacrylate. Hydrogels of this nature are further described in International Publication No. WO 2007/146722. Alternatively, for certain of these embodiments, the hydrogel comprises an ionically crosslinked alginate. Alternatively, for certain of these embodiments, the hydrogel comprises the reaction product of a hydrophilic polyol and a polyisocyanate. Hydrophilic polyols include polyethylene glycols and the like. Polyisocyanates include 2,4-tolylene diisocyanate, 2,6-tolylene diisocyanate, diphenylmethane diisocyanate (MDI), and the like. Alternatively, for certain of these embodiments, the hydrogel comprises a crosslinked guar gum.

The hydrogels of the present invention may have a equilibrium water concentration of 5-95% water. That is, when placed in deionized water and allowed to reach an equilibrium water concentration (which is typically done by allowing it to soak submerged for 24 hrs) the weight percent water in the composition will be at least 5%, preferably at least 10%, and most preferably at least 20%. The equilibrium water concentration is preferably not greater than about 95% so that the gel will have sufficient integrity. Preferably the equilibrium water content is not more than 90% by weight and most preferably not more than 85% by weight.

The nanoparticles described herein, having thiol-containing groups or nitrosothiol-containing groups attached to the surface of the nanoparticles, may be distributed in a hydrogel by mixing the nanoparticles in a hydrogel monomer and polymerizing the monomer. The resulting polymer may further be crosslinked by known radiation or chemical crosslinking methods or by ionic crosslinking. The hydrogel monomer may be a single monomer or a mixture of monomers, for example, as described above. Prior to polymerizing the monomer, a portion or essentially all of the water and any other volatile component, such as a lower alcohol, may be removed under reduced pressure. Natural hydrogel forming polymers may be added to a dispersion of the nanoparticles in an aqueous system to provide a dispersion of the nanoparticles in a natural polymer hydrogel. Alternatively, a dispersion of nanoparticles may be added to a solution or dispersion of a natural hydrogel forming polymer followed by covalent or ionic crosslinking. Polyanionic polymers, for example, alginates, may be crosslinked with multivalent metal ions such as Ca⁺⁺, Mg⁺⁺, Zn⁺⁺, Fe⁺⁺, Fe⁺⁺⁺, Al⁺⁺⁺, or with polyamines. Polycationic polymers may be crosslinked with polyanionic compounds such as polycarboxylic, polysulfonic, polysulfate, polyphosphonate, and polyphosphate oligomers.

For certain embodiments, including any one of the above embodiments other than those where the nanoparticles, whether nanoparticles having an external surface comprising solid amorphous silica or solid amorphous silica nanoparticles, are dispersed in an aqueous system, the nanoparticles are dispersed in a hydrogel monomer or a hydrophilic polyol. For these embodiments, preferably, the nanoparticles comprise stabilizing groups attached to the surface of the nanoparticles which stabilize the dispersion in the monomer or polyol. Such dispersions can be prepared by combining a dispersion of the nanoparticles in an aqueous system with the monomer(s) or polyol(s), and removing some or all of the water and optionally any other volatile components.

For certain embodiments, including any one of the above embodiments where the composition further comprises a hydrogel, the hydrogel is a plurality of hydrogel beads. Such hydrogel beads are further described in WO 2007/146722.

For certain of these embodiments, including any one of the above embodiments where the composition further comprises a hydrogel, the hydrogel is substantially dehydrated. Having a reduced water content can help increase the storage stability of the present compositions by reducing premature release of NO during storage.

For certain embodiments, including any one of the above compositions, the composition is transparent. As indicated above, this may be determined by viewing the composition through a path length of 1 cm or less. Because of the particle size range of the nanoparticles used in the present compositions, and because these nanoparticles are substantially not agglomerated, the nanoparticles cause minimal light scattering, thereby making it possible for the compositions to be transparent. This allows visual observation of a structure, such as a wound, which is covered by the composition.

In another embodiment, there is provided a medical device comprising a surface and any one of the above embodiments of a composition for releasing nitric oxide adjacent the surface. For example, the composition for releasing nitric oxide can be coated onto a surface of the medical device. The surface can be that of a sheet, a film, a woven, knit, or nonwoven fabric, a fiber, a filament, a thread (made of multiple fibers or filaments, or a monofilament), a tube, or the like. The surface can be coated with one or more coatings, for example, an adhesive coating, a primer coating, or a coating containing a coupling agent, prior to coating the composition for releasing nitric oxide.

For certain embodiments, including any one of the above embodiments of the medical device, the composition for releasing nitric oxide is any one of the compositions described supra, which comprises a first part and a second part. For certain of these embodiments, any one of the above embodiments of the first part is adjacent the surface of the medical device, for example, coated on the surface of the medical device. Any one of the above embodiments of the second part, which contains the nitrite source, can be contained and positioned adjacent the first part for ease of combining with the first part. Such combination can be done by the clinician prior to application or it can occur without deliberate mixing, i.e., it can occur passively as the dressing hydrates. In a dressing where the nitrite source and thiol are separated until use it is preferred that the thiol be in molar excess to ensure low or no residual nitrite.

For certain embodiments, including any one of the above embodiments of the medical device, the device is selected from the group consisting of a wound dressing, wound contact layer, a wound filler, a medical tape, a surgical thread, a vascular graft, a stent, and a catheter.

For certain embodiments, including any one of the above embodiments of the medical device, the device is a wound dressing. For certain embodiments, the wound dressing comprises a backing with the composition for releasing nitric oxide adjacent a surface of the backing.

Suitable backing materials for the backing include, for example, nonwoven fibrous webs, woven fibrous webs, knits, films, and the like. For certain embodiments, the backing is a translucent or transparent polymeric elastic film. The backing can be a high moisture vapor permeable film backing U.S. Pat. No. 3,645,835 (Hodgson) describes methods of making such films and methods for testing their permeability. For certain embodiments, preferred suitable backing materials are elastomeric polyurethane, co-polyester, or polyether block amide films, which are described in U.S. Pat. No. 5,088,483 (Heinecke) and U.S. Pat. No. 5,160,315 (Heinecke et al.). These films have properties of resiliency, high moisture vapor permeability, and transparency.

For certain embodiments, the wound dressing comprises a hydrogel layer comprising the composition for releasing nitric oxide, a backing layer, and an adhesive layer on the backing layer facing the hydrogel layer. For certain embodiments, the adhesive layer and backing layer can form a perimeter around the hydrogel layer where the hydrogel layer does not cover the entire adhesive layer. The perimeter formed by the adhesive layer and backing layer can keep the hydrogel layer properly positioned, for example, with respect to a wound, and also helps maintain a sterile environment around the application surface. The hydrogel layer may be continuous or be coated in a pattern with areas of hydrogel and areas of no hydrogel, e.g., patterns such as dots, grids, etc.

For certain embodiments, the adhesive layer and backing layer can be very thin, and flexible. If this adhesive layer and backing layer are not properly supported during application they may fold over and adhere to themselves, preventing proper application over a surface. The adhesive layer and backing layer are optionally supported by a removable carrier layer attached to the top face (side opposite the side with the adhesive and hydrogel layers) of the backing layer. Optionally, a release liner is provided to contact the adhesive and the hydrogel layer. Both the release liner and the backing layer coated with the adhesive layer extend beyond the edges of the hydrogel layer.

The carrier layer is generally substantially more rigid than the backing layer to prevent the backing layer from improperly wrinkling during application to a surface. The carrier layer can be heat-sealable to the backing layer with or without a low adhesion coating. Suitable carrier layers include, for example, polyethylene/vinyl acetate copolymer-coated papers and polyester films. The carrier layer may include perforations to aid in separating portions of the carrier layer after application of the dressing to a surface.

Various pressure sensitive adhesives can be used to form the adhesive layer on the backing layer to make it adhesive. The pressure sensitive adhesive is usually reasonably skin compatible and “hypoallergenic”, such as the acrylate copolymers described in U.S. Pat. No. RE 24,906 (Ulrich). For certain embodiments, useful adhesives are a 97:3 isooctyl acrylate:acrylamide copolymer or 70:15:15 isooctyl acrylate:ethyleneoxide acrylate:acrylic acid terpolymer described in U.S. Pat. No. 4,737,410 (Kantner). Additional useful adhesives are described in U.S. Pat. No. 3,389,827 (Abere et al.); U.S. Pat. No. 4,112,213 (Waldman); U.S. Pat. No. 4,310,509 (Berglund); and U.S. Pat. No. 4,323,557 (Rosso et al.). Inclusion of medicaments or antimicrobial agents in the adhesive is also contemplated, as described in U.S. Pat. Nos. 4,310,509 and 4,323,557.

The adhesive layer can be coated on the backing layer by a variety of processes, including, direct coating, lamination, and hot lamination.

Suitable release liners for use as described herein can be made of kraft papers, polyethylene, polypropylene, polyester or composites of any of these materials. The films are preferably coated with release agents such as fluorochemicals or silicones. For example, U.S. Pat. No. 4,472,480 describes low surface energy perfluorochemical liners. Fluoropolymer coated polyester films are commercially available from 3M (St. Paul, Minn.) under the brand “ScotchPak™” release liners. Examples of commercially available silicone coated release papers are POLYSLIK™, silicone release papers available from Rexam Release (Bedford Park, Ill.) and silicone release papers supplied by LOPAREX (Willowbrook, Ill.). Siliconized polyethylene terephthalate films are commercially available from H. P. Smith Co.

Exemplary dressing constructions such as those described herein include FIGS. 1-4, described at col. 2, line 64 to col. 6, line 28, in U.S. Pat. No. 6,436,432; FIGS. 1 and 1A-1C, described at col. 3, line 65 to col. 4, line 43 in U.S. Pat. No. 6,903,243; FIG. 1, described at col. 19, line 53 to col. 20, line 9; and FIGS. 1-10, described at page 4, paragraph 0046 through page 6, paragraph 0077 in US 2004/0133143.

For certain embodiments, including any one of the above embodiments of the medical device where the composition for releasing nitric oxide is any one of the compositions described supra, which comprises a first part and a second part, the first part and the second part of the composition for releasing nitric oxide are each separately enveloped by a barrier material, wherein preferably the barrier material is impervious to water. Suitable barrier materials are known and generally are multi-layer films which include at least one polymer layer, such as polyethylene, and a metal foil, metallized layer, fluorinated polymer layer, or metal oxide layer. For certain of these embodiments, the enveloped first part and the enveloped second part are separated by a breachable barrier, which when breached allows the second part to combine with the first part.

In another embodiment, there is provided a product comprising a medical device according to any one of the above embodiments of a medical device and a package impervious to water, water vapor, ultraviolet light, and visible light, wherein the composition for releasing nitric oxide is enveloped by the package. Preferably, the package is hermetically sealed. For certain embodiments, the entire medical device is enveloped by the package. Alternatively, a limited portion of the medical device is enveloped by the package, wherein the limited portion includes the composition for releasing nitric oxide. Suitable package materials include, for example, a metal foil coated with a protective and/or heat sealable polymeric layer. In one example, the package material is a polyolefin coated aluminum foil. Suitable barrier layers and barrier constructions for packaging materials are further described, for example, in U.S. Pat. No. 7,261,701.

In another embodiment, there is provided method of treating a subject with nitric oxide, the method comprising: providing a composition for releasing nitric oxide, including any one of the above embodiments of a composition for releasing nitric oxide; contacting the subject with the composition; and releasing nitric oxide at a location where the composition contacts the subject.

In another embodiment, there is provided method of treating a subject with nitric oxide, the method comprising: providing any one of the above embodiments of a medical device comprising a surface and any one of the above embodiments of a composition for releasing nitric oxide adjacent the surface; contacting the subject with the medical device; and releasing nitric oxide at a location where the medical device contacts the subject.

For certain embodiments, including any one of the above embodiments of the method of treating a subject with nitric oxide, the composition for releasing nitric oxide is any one of the compositions described supra, which comprises a first part and a second part. For such embodiments, the method of treating the subject with nitric oxide further comprises combining the first part of the composition with the second part of the composition to provide a composition wherein nitrosothiol-containing groups are attached to the surface of the nanoparticles.

For certain embodiments, including any one of the above methods for treating a subject with nitric oxide, the method further comprises activating the release of the nitric oxide from the composition. For certain of these embodiments, activating includes exposing the composition to an activating agent selected from the group consisting of an aqueous composition, a body fluid, a thiol-containing compound, an ascorbate salt, visible light, ultraviolet light, and a combination thereof.

The release of nitric oxide from the composition can be activated or the rate of release can be substantially increased by increasing the water content of the composition, such as by exposing the composition to an aqueous composition or to a body fluid. The aqueous composition contains at least 50 percent by weight of water, preferably at least 75 or at least 90 percent by weight of water. The body fluid also contains water and can be a wound exudate, blood, an aqueous blood component, mucus, urine, or the like.

The release of nitric oxide from the composition can also be activated or the rate of release can be substantially increased by exposing the composition to a thiol-containing compound, an ascorbate salt, visible light, ultraviolet light, or a combination thereof. The thiol-containing compound is preferably a small molecule, for example a thiol with molecular weight of not more than about 350, preferably not more than about 200 or not more than about 150. Such thiol-containing compounds may penetrate the stabilizing groups on the nanoparticles and reach the nitrosothiol groups, causing disulfide formation and release of NO. Suitable thiol-containing compounds include, for example, cysteine, penicillamine, glutathione, salts thereof, and the like. Thiol-containing compounds having very low molecular weights, for example, a molecular weight less than about 75, may be avoided because of excessive volatility and odor. The thiol-containing compound can be included in the above aqueous composition.

An ascorbate salt, such as sodium ascorbate, can be used to increase release of nitric oxide from the composition. It is believed that the ascorbate reacts directly with the nitrosothiol groups, releasing NO, and forming the thiol-containing group on the nanoparticles and dehydroascorbate. The ascorbate salt can be included in the above described aqueous compositions. Other antioxidants also may be useful.

The —S—NO functional group has absorption maxima at 550 to 600 and 330 to 350 nanometers. Accordingly when visible and/or ultraviolet light is used to activate or increase the rate of release of NO, the visible light preferably includes wavelengths in the range of 550 to 600 nanometers, and the ultraviolet light preferably includes wavelengths in the range of 330 to 350 nanometers.

For certain embodiments, including any one of the above embodiments which includes activating the release of the nitric oxide from the composition, and activating includes exposing the composition to an activating agent, activating is carried out by exposing the composition to a body fluid, and wherein the body fluid is a wound exudate. Preferably, the composition is in intimate contact with a body tissue to allow body fluid to easily contact the composition.

Where the composition for releasing nitric oxide comprises a first part and a second part, preferably the first and second parts are combined prior to activating the release of NO.

In another embodiment, there is provided a kit comprising any one of the above embodiments of a composition for releasing nitric oxide and an activating agent.

In another embodiment, there is provided a kit comprising any one of the above embodiments of a medical device comprising a surface and any one of the above embodiments of a composition for releasing nitric oxide adjacent the surface; and an activating agent.

Suitable activating agents for use in the above kits include, for example, an aqueous composition, a thiol-containing compound, an ascorbate salt, or a combination thereof as described supra. When light, whether visible and/or ultraviolet, is to be used as an activating agent, the kit can include a light source which emits visible and/or ultraviolet light as described supra.

In one embodiment, the kit contains a device having nanoparticles with thiol-containing groups attached to the surface of the nanoparticles and a nitrite source physically separated, for example, separately contained. These may be mixed prior to use, or they may mix due to diffusion and dissolution by an aqueous composition diffusing into a coated device, for example, wherein a composition for releasing nitric oxide is coated onto a surface of the medical device as described supra.

In another embodiment, there is provided a method of making a composition for releasing nitric oxide, the method comprising: providing solid amorphous silica nanoparticles; bonding thiol-containing groups to an exterior surface of the solid amorphous silica nanoparticles; bonding stabilizing groups comprising hydrophilic groups to the exterior surface of the solid amorphous silica nanoparticles; and nitrosylating the thiol-containing groups to provide solid amorphous silica nanoparticles having an exterior surface wherein nitrosothiol-containing groups are attached to the surface.

The solid amorphous silica nanoparticles useful in the above method of making the present compositions can be provided by obtaining such nanoparticles from a commercial source as indicated supra. Alternatively, the solid amorphous silica nanoparticles for use in the above method of making a composition for releasing nitric oxide are provided by condensing a silicate under basic conditions as described supra. For certain of these embodiments, the silicate is selected from the group consisting of sodium silicate, tetraethyl orthosilicate, and tetramethyl orthosilicate.

For certain embodiments, including the above method of making a composition for releasing nitric oxide, bonding the thiol-containing groups to an exterior surface of the solid amorphous silica nanoparticles is carried out by reacting the alkoxysilyl portion of a compound, which includes alkoxysilyl and thiol groups, with the silica nanoparticles. This can be carried out as described supra.

For certain embodiments, including any one of the above methods of making a composition for releasing nitric oxide, bonding the stabilizing groups comprising hydrophilic groups to the exterior surface of the solid amorphous silica nanoparticles is carried out by reacting the alkoxysilyl portion of a compound, which includes alkoxysilyl and hydrophilic groups, with the silica nanoparticles. This can be carried out as described supra.

For certain embodiments, including any one of the above methods of making a composition for releasing nitric oxide, nitrosylating the thiol-containing groups is carried out by reacting the particles with thiol-containing groups with a nitrite salt under acidic conditions. A molar amount of nitrite equivalent to or greater than the molar amount of thiol-containing groups present may be used. Suitable nitrite salts include, for example, sodium, potassium, and ammonium salts. In an alternative embodiment, nitrous acid or a combination of nitrous acid and a nitrite salt is used for nitrosylating the thiol-containing groups.

The use of an excess of nitrite has been found to reduce the rate of NO release, and can be used to add additional stability to the nitrosothiol-containing groups on the nanoparticles. For example, a molar ratio of nitrite to thiol-containing groups of 1.5:1 or 2:1 or more can be used. For any one of the above described embodiments which includes a composition for releasing NO, the composition can include such an excess of nitrite. For certain embodiments, for each mole of nitrosothiol-containing groups, 0.5 mole, 1 mole, or more of nitrite is present.

For certain embodiments, including any one of the above methods of making a composition for releasing nitric oxide, the method further comprises dispersing the solid amorphous silica nanoparticles, having an exterior surface wherein nitrosothiol-containing groups are attached to the surface, in an aqueous system. Suitable aqueous systems include those described supra.

For certain embodiments, a metal ion sequestering agent is added in order to inhibit rapid breakdown of nitrosothiols, catalyzed by metal ions such as ferric ion. Suitable metal ion sequestering agents include chelators such as ethylenediamine-N,N,N′,N′-tetraacetic acid (EDTA), 1,3-diaminopropane-N,N,N′,N′-tetraacetic acid, N,N-bis(2-hydroxethyl)glycine (DHEG), ethylenediamine-N,N′-bis(methylenephosphonic acid) (EDDPO), iminodiacetic acid (IDA), nitrilotriacetic acid (NTA), dipicolinic acid (DPA) as well as salts of the forgoing acids. Also immobilized chelators, such as CHELEX 100 resin (Bio-Rad Laboratories, Hercules, Calif.) can be used. Metal ions may also be sequestered by removing them from the liquid phase by precipitation or adsorption.

The features and advantages of this invention are further illustrated by the following examples, which are in no way intended to be limiting thereof. The particular materials and amounts thereof recited in these examples, as well as other conditions and details, should not be construed to unduly limit this invention. Unless otherwise indicated, all parts and percentages are on a weight basis, all water is deionized water, and all molecular weights are weight average molecular weight.

### EXAMPLES

### Abbreviations, Descriptions, and Sources of Materials

| Abbreviation | Description and Source |
|---|---|
| NALCO 2326 sol | 5 nanometer solid amorphous silica |
| | nanoparticles dispersed in water at 17 |
| | weight percent nanoparticles (available |
| | from Nalco Co., Naperville, Il) |
| Ethanol | 200 proof (Aldrich Chemical Co., |
| | Milwaukee, WI) |
| MPTMS | 3-mercaptopropyltrimethoxysilane |
| | (available as SIM6476.0 from Gelest Inc., |
| | Morrisville, PA) |
| PEOTES | a poly(ethylene glycol)triethoxysilane with |
| | a molecular weight of about 500 (available |
| | under tradename, SILQUEST A1230, from |
| | Momentive Performance Materials, Wilton, |
| | CT) |
| GAPTES | N-(3-triethoxysilylpropyl)gluconamide, |
| | 50% wt in ethanol (available as SIT 8189.0 |
| | from Gelest Inc., Morrisville, PA) |
| Sodium Nitrite | NaNO₂ (available from Aldrich Chemical |
| | Co., Milwaukee WI)) |
| HEMA | 2-hydroxyethyl methacrylate (Mitsubishi |
| | Rayon Co., Ltd., Tokyo, Japan) |
| HEA | 2-hydroxyethyl acrylate (available from |
| | aldrich Chemical Co., Milwaukee, WI) |
| M-PEG | Poly(ethylene glycol) methyl ether acrylate |
| | (Aldrich Chemical Co., Milwaukee, WI) |
| MAA-PEG | Poly(ethylene oxide-ran-propylene oxide) |
| | dimethacrylate prepared as described in |
| | WO 03/086493, number average molecular |
| | weight about 20,000 |
| EDMAB | ethyl 4-(N,N-dimethylamino)benzoate |
| | (Quantacure EPD, Biddle Sawyer Corp., |
| | New York, N.Y.) |
| CPQ | Camphorquinone (Aldrich Chemical Co.) |

### Example 1

### Preparation of Solid Amorphous Silica Nanoparticles with Nitrosothiol Groups

NALCO 2326 sol (50 g) was combined with ethanol (50 g) and MPTMS (0.838 g) in a bottle. The resulting mixture was purged with nitrogen for 15 minutes with constant stirring. The bottle was then sealed and heated at 70° C. in a water bath under constant stirring for 8 hours to provide nanoparticles dispersed in water and ethanol (1.2:1), with 3-mercaptopropylsilyl groups attached to the nanoparticles (sol A).

A portion of the above sol A (75 g) was combined with PEOTES (5.656 g). The resulting mixture was purged with nitrogen and heated for 8 hours at 70° C. with constant stirring to provide nanoparticles dispersed in water and ethanol, with poly(alkylene oxide)silyl groups also attached to the nanoparticles (sol B).

A portion of the above sol B (25 g) was placed in a bottle, and 1N hydrochloric acid was added to adjust the pH of the sol to 3. Sodium nitrite (85.9 mg; 1.2 mol sodium nitrite and 1.2 mol hydrochloric acid per mol of MPTMS used for the amount of nanoparticles in the portion of sol B) was added to the mixture. The resulting nitrosylated sol (sol C) was red, indicating conversion of the sulfhydryl groups to nitrosothiol groups. Sol C was stored under nitrogen in sealed vials protected from light and kept at 5° C.

### Example 2

### Release of NO from Nitrosylated Sol

A nitrosylated sol was prepared essentially as described in Example 1 and diluted 1:34 with water. The pH of the resulting dilute sol was 4.5. The ratio of nitrite to thiol(sulfhydryl) groups on the nanoparticles was 1.7:1. Using a Hewlett-Packard 8452A Spectrophotometer (Agilent Technologies, Santa Clara, Calif.), the absorbance of the sol was measured serially over 16 days at 336 nanometers, the UV absorbance peak wavelength of the nitrosothiol groups. Over this time period, the sol was exposed to ambient fluorescent lighting at a temperature of 20° C. Each of the absorbance values was normalized with respect to the initial absorbance. The results shown in Table 1 below indicate that NO was released over the 16 day time period as seen by the decay of the nitrosothiol group absorbance, and that at day 16, the sol maintained approximately 30 percent of the absorbance due to the remaining nitrosothiol groups.

TABLE 1
Normalized Nitrosothiol Group Absorbance from 0 to 16
Days Under Fluorescent Lighting and Percent NO Released

| Time | Absorbance (A)/Absorbance | |
|---|---|---|
| (Days) | At 0 Days (A₀) | Percent NO Released¹ |
| 0 | 1 | 0 |
| 1 | 0.87 | 13 |
| 2 | 0.69 | 33 |
| 5 | 0.53 | 47 |
| 6 | 0.48 | 52 |
| 8 | 0.42 | 58 |
| 12 | 0.36 | 64 |
| 16 | 0.30 | 70 |

¹(A₀ − Aₙ)/A₀ × 100 = Percent NO released as a function of decrease in absorbance at 336 nm.

### Example 3

### Ascorbate Induced Accelerated Release of NO from Nitrosylated Sol

Separate nitrosylated sols were prepared as in Example 2. Sodium ascorbate was added to one sol at a concentration of 1.2 mM. The absorbance of each of the sols was measured serially over 7 days as in Example 2. Over this time period, the sols were protected from light, and held at 20° C. Each of the absorbance values was normalized with respect to the initial absorbance and the percent NO released was calculated as in Example 2. The results shown in Table 2 below indicate that the ascorbate causes a significant increase in the NO release rate.

TABLE 2
Release of NO from Nitrosylated Sols With and Without Ascorbate

| | |
|---|---|
| Time | Percent NO Released |

| (Days) | With Ascorbate | Without Ascorbate |
|---|---|---|
| 0 | 0 | 0 |
| 1 | 38.6 | 23.7 |
| 4 | 63.2 | 44.8 |
| 7 | 67.4 | 50.2 |

### Example 4

### Thiol Induced Accelerated Release of NO from Nitrosylated Sol

Separated nitrosylated sols were prepared as in Example 2. 1-Propanethiol was added to one sol at a concentration of 0.13 M. The absorbance of each of the sols was measured serially over 6 days as in Example 2. Over this time period, the sols were protected from light, and held at 5° C. Each of the absorbance values was normalized with respect to the initial absorbance and the percent NO released was calculated as in Example 2. The results shown in Table 3 below indicate that the 1-propanethiol causes a very large increase in the NO release rate.

TABLE 3
Release of NO from Nitrosylated Sol
in the Presence of 1-Propanethiol

| | |
|---|---|
| Time | Percent NO Released |

| (Days) | With 1-Propanethiol | Without 1-Propanethiol |
|---|---|---|
| 0 | 0 | 0 |
| 1 | 66.3 | 2.3 |
| 4 | 84.2 | 7.6 |
| 6 | 91.0 | 11.0 |

### Example 5

### Thermal Stability of Nitrosylated Sol

Nitrosylated sols were prepared as in Example 2. Absorbance values of the sols were measured serially over 21 days as in Example 2. Over this time period, the sols were protected from light, but held at 20° C., 5° C., and −20° C., respectively. Each of the absorbance values was normalized with respect to the initial absorbance and the percent NO released was calculated as in Example 2. The results shown in Table 4 below indicate that much less NO was released in the dark compared with the amount of NO released under light in Example 2. The results also show that NO release is greatly reduced at lower temperatures. Protection from light and reduced temperatures can, therefore, enhance storage stability.

TABLE 4
Percent NO Released at 0 to 21 Days in the
Dark at 20° C., 5° C., and −20° C.

| Time | Percent NO Released | | |
|---|---|---|---|
| (Days) | 20° C. | 5° C. | −20° C. |
| 0 | 0 | 0 | 0 |
| 1 | 10 | 5.1 | 1.4 |
| 2 | 16.1 | 8.7 | 5.0 |
| 5 | 29.8 | 13.6 | 6.0 |
| 6 | 32.4 | 16.0 | 5.6 |
| 8 | 36.4 | 18.3 | 7.5 |
| 12 | 43.3 | 20.8 | 8.8 |
| 16 | 47.8 | 24.8 | 11.0 |
| 21 | 50.9 | 27.9 | 11.9 |

### Example 6

### Effect of Excess Nitrite on Release of NO from Nitrosylated Sol

Separate nitrosylated sols were prepared as in Example 2 with the molar ratio of nitrite ion to MPTMS at 1:1 and 1.7:1, respectively. The uv absorbance of each of the sols was measured serially over 21 days and normalized with respect to the initial absorbance, and the percent NO release was calculated as in Example 2. Over this time period, the sols were protected from light and held at 5° C. The results shown in Table 5 below indicate that much less NO was released when an excess of nitrite was used. The presence of excess nitrite ion can, therefore, enhance storage stability.

TABLE 5
Percent NO Released at 0 to 21 Days in the Dark with
Ratios of Nitrite to Starting Thiol of 1:1 and 1.7:1

| | |
|---|---|
| Time | Percent NO Released |

| (Days) | 1:1 (nitrite/thiol) | 1.7:1 (nitrite/thiol) |
|---|---|---|
| 0 | 0 | 0 |
| 1 | 11.7 | 4.5 |
| 2 | 17.2 | 8.7 |
| 5 | 23.7 | 13.6 |
| 6 | 27.1 | 16.0 |
| 8 | 31.5 | 18.3 |
| 12 | 35.4 | 20.8 |
| 16 | 38.1 | 24.8 |
| 21 | 41.0 | 27.9 |

### Example 7

### Nitrosylated Nanoparticles in Hydrogel

Nitrosylated sol prepared as in Example 1 (25 g) was dialyzed in pure water using 12,000-14,000 molecular weight cutoff membrane tubing (SPECTRA/POR MOLECULARPORO, Spectrum Laboratories, Inc., Rancho Dominguez, Calif.) prior to the nitrosylation step and then added to M-PEG (8 grams), and at least about 90% of the water/ethanol solvent in resulting mixture was removed under reduced pressure in a rotary evaporator. The nitrosothiol groups were still present as indicated by the red color of the resulting sol. This sol (1.7 g) was then combined with MAA-PEG (0.90 g), HEMA (0.22 g), CPQ (25 mg) and EDMAB (25 mg). The resulting mixture was placed in rectangular mold with a depth of 0.32 cm and photopolymerized for 5 minutes under a DENTAL BLUE light (available from 3M ESPE, St. Paul, Minn.) equipped with a 455 nanometer cutoff filter to prevent photoinitiated NO release. The resulting non-hydrated hydrogel retained the red color of the nitrosylated sol, indicating the presence of the nitrosothiol groups. This color was stable for weeks with the hydrogel protected from light at 5° C. When the hydrogel was immersed in water, the red color diminished significantly within hours.

### Example 8

### Antimicrobial Effect of NO Release from Nitrosylated Nanoparticles

Nitrosylated sols were prepared essentially as in Example 1, except that the sols were dialyzed in pure water using 12,000-14,000 molecular weight cutoff membrane tubing (SPECTRA/POR MOLECULARPORO, Spectrum Laboratories, Inc., Rancho Dominguez, Calif.) prior to the nitrosylation step. Non-nitrosylated sols were prepared as in Example 1 (sol B) and dialyzed as above. To each of the sols was added cysteine hydrochloride (6 mg/g sol) to facilitate release of NO from nitrosothiol groups, if present. Portions of each sol were diluted 10 fold, 100 fold, 1000 fold, and 10,000 fold. Undiluted and diluted sols were inoculated with S. aureus and P. aeruginosa and incubated at 37° C. for two weeks. Survival of the challenge organisms was assessed at regular intervals over the two week period by standard plate counting methods. Results of the survival assessments for S aureus are shown in Table 6 below, in which the normalized minimum inhibitory concentration (NMIC) of nitrosylated nanoparticles necessary for total kill of S. aureus are shown. No significant antimicrobial effect against P. aeruginosa was observed for any of the tested sols.

TABLE 6
Total Kill of S. aureus as a Function of Normalized Nitrosylated
and Non-Nitrosylated Nanoparticle Concentrations

| Normalized | Thiolated Nanoparticles | Nitrosylated Nanoparticles |
|---|---|---|
| Concentration | (days to total kill) | (days to total kill) |
| 1 | 3 | 3 |
| 0.1 | 7 | 7 |
| 0.01 | 14 | 10 |
| 0.001 | 14 | 10 |
| 0.0001 | Total kill not observed | Total kill not observed |

### Example 9

### Two-Part Composition with Alginate Hydrogel

A thiolated nanoparticle sol (non-nitrosylated) was prepared as described above in Example 1 (sol B) and diluted 1:1 with deionized water. Sodium alginate (Aldrich Chemical Co.) was added to the sol at about 3 percent by weight. The resulting alginate/nanoparticle sol mixture was injected through a filling tab into compartment A of a two-compartment foil container equipped with filling tabs for each compartment, leaving a portion of the volume of compartment A unfilled. Such containers are available from 3M ESPE, St. Paul, Minn. Hydrochloric acid (˜30 mL 1 N, Malinkrodt Specialty Chemicals, Paris, Ky.) was then added to compartment A through the filling tab to provide an acidified alginate gel containing non-nitrosylated nanoparticles dispersed throughout the gel. The filling tab was folded to seal compartment A. A saturated solution of sodium nitrite was injected through a filling tab into compartment B of the container, and the filing tab was folded to seal compartment B. The contents of compartment B was emptied into compartment A by squeezing compartment B, causing the foil layers separating the compartments to separate, allowing the nitrite solution to combine with the alginate gel. The resulting alginate gel containing nitrosylated nanoparticles was observed when the top of compartment A was peeled away, revealing the red nitrosylation reaction product.

### Example 10

### Effects of Added Cysteine HCl and Copper Ions on NO Release from Nitrosylated Sols

A nitrosylated sol was prepared essentially as in Example 1, except that the sol was dialyzed in pure water as in Example 8. The resulting sol was diluted 1:29 with water. To each of four 1.5 mL portions of the resulting diluted sol was added 40 μL water, 40 μL 18 mM cupric bromide, 40 μL 47 mM cysteine hydrochloride, 40 μL 47 mM cysteine hydrochloride plus 40 μL 18 mM cupric bromide, respectively, to provide the following sols: control, 0.5 mM Cu⁺⁺, 1.25 mM cysteine HCl, 1.25 mM cysteine HCl with 0.5 mM Cu⁺⁺. Presence of Cu⁺⁺ is known to cause decomposition of the S—NO group where the Cu⁺⁺ is first reduced to Cu⁺, for example, in the presence of a thiol, and the Cu⁺ then reacts with the S—NO group, releasing NO and forming a thiolate anion and Cu⁺⁺ (which can be reduced to Cu⁺ by the thiolate). The UV absorbance (A) of the sols was measured serially over 4 days at 336 nanometers as in Example 2. Over this time period, the sols were protected from light and held at 5° C. The amount of NO released was calculated by first calculating the concentration of S—NO groups using Beer's Law (A=εbc), where the extinction coefficient ε is 900 (M cm)⁻¹, and subtracting the resulting concentration from the concentration determined initially. The results are shown in Table 7. The presence of cysteine HCl sharply increased the rate of S—NO consumption during the first 12 hours. Over the remaining time period, the rate of S—NO consumption in the presence of cysteine HCl was similar to that of the control and Cu⁺⁺ containing sols.

Thiols are known to react with nitrosothiols, releasing NO and forming disulfides. The sharp initial increase in the rate of NO release in the presence of cysteine HCl, therefore, is believed to be the result of reaction between the cysteine and the nitrosothiol groups on the nanoparticles, forming disulfide groups.

The slow release of NO in the control sols is believed to be the result of simple dissociation of nitrosothiol groups, resulting in sulfhydryl(thiol) groups formation. The presence of Cu⁺⁺ (without the cysteine) had no effect on nitrosothiol group degradation. These findings provide evidence consistent with the nitrosothiol groups on the nanoparticles being sterically shielded or otherwise stabilized against intra- and inter-particle disulfide formation. In the case of a small thiol molecule, such as cysteine or 1-propanethiol, the small thiol can penetrate the shielding of the stabilizing groups and displace NO, forming disulfide groups.

TABLE 7
Effects of Cysteine HCl and Cu⁺⁺ on NO
Release from Nitrosylated Sols

| | NO Released (mmol/g solids in sol) | | | |
|---|---|---|---|---|
| | | | | 1.25 mM |
| Time | Control | | 1.25 mM | Cysteine HCl |
| (Days) | Sol | 0.5 mM Cu⁺⁺ | Cysteine HCl | and 0.5 mM Cu⁺⁺ |
| 0 | 0 | 0 | 0 | 0 |
| 0.25 | 0.081 | 0.083 | 0.421 | 0.536 |
| 1 | 0.202 | 0.206 | 0.463 | 0.660 |
| 2 | 0.354 | 0.349 | 0.582 | 0.836 |
| 3 | 0.525 | 0.507 | 0.672 | 0.924 |
| 6 | 0.963 | 1.015 | 0.945 | 1.198 |
| 8 | 1.392 | 1.425 | 1.315 | 1.377 |

### Example 11

### Stability of Thiolated Sols

Thiolated sols were prepared as in Example 1 (sol B). The sols were stored in the dark and at room temperature for a period of 10 months. The sols were found to maintain their optical clarity during this time. After 10 months, the thiolated nanoparticles in the sols were reacted with nitrite as in Example 1 and found to form nitrosothiol groups as well as prior to the storage period. This is surprising in view of the disulfide formation known to occur (and normally avoided with reducing agents) with small molecules and polymers containing thiol groups. The stability of the thiol groups on the nanoparticles makes the two-part compositions described above possible.

### Example 12

### Preparation of Solid Amorphous Silica Nanoparticles with Glucose and Nitrosothiol Groups

### Preparation of Thiolated Nanoparticles:

100 g of NALCO 2326 sol (17% wt, Nalco, Naperville, Ill.) was charged into a 100 mL, 3-necked round bottom flask fitted with a glass stopper, condenser, and a thermometer. The sol was continuously stirred using a magnetic stirrer. 1.25 mL of concentrated nitric acid was added drop-wise to reach a pH of 1 to 3. Using a heating mantle, the sol temperature was increased to 70° C. (+/−5° C.) and combined with 24.72 g N-(3-triethoxysilylpropyl)gluconamide (50% wt in ethanol, Gelest Inc., Morrisville, Pa.). The resulting sol was maintained at temperature for 8 hours, and then 1.52 g of 3-mercaptopropyltrimethoxysilane (Alfa Aesar, Ward Hill, Mass.) was added. The resulting sol mixture was maintained at temperature with stirring for an additional 8 hours to provide a thiolated nanoparticle sol, containing nanoparticles with 3-mercaptopropylsilyl and 3-gluconamidopropylsilyl groups.

### Nitrosylation of Thiolated Nanoparticles:

The above glucose-stabilized thiolated nanoparticle sol (5 g) was placed in a vial, and 1N hydrochloric acid was added to adjust the pH of the sol to 3. Then sodium nitrite (0.027 g) was added to the sol and vortexed to mix. The resulting nitrosylated sol was red, indicating conversion of the sulfhydryl groups to nitrosothiol groups. This sol was stored in a sealed vial protected from light and kept at 5° C.

### Example 13

### Generation of Nitric Oxide from Nanoparticles Incorporated in a Shaped Hydrogel

### Preparation of Thiolated Nanoparticles:

The following was placed in a heated round bottom three neck flask fitted with a reflux condenser, a thermometer, and a nitrogen purge line:

200 mL NALCO 2326 sol containing silica nanoparticles at 15% by weight in water (Nalco, Naperville, Ill.)

200 g ethanol (200 proof)

3.35 g 3-mercaptopropyltrimethoxysilane (Alfa Aesar, Ward Hill, Mass. 01835). The flask contents were continually stirred using a magnetic stirrer. Before heating, the flask was purged with nitrogen for 15 minutes to remove oxygen. It was then heated to 70° C. for 7 hours. During heating the temperature varied from 65 to 75° C. The flask was cooled to room temperature, and PEOTES (30.2) g was added. The flask was again purged with nitrogen for 15 minutes and then heated to 70° C. (+/−5° C.) for 7 hours. The volume of the resulting mixture was reduced to about 100 mL by rapid vacuum distillation at 70° C., using a Buchi R110 ROTAVAPOR, to provide a poly(ethylene glycol) stabilized thiolated nanoparticle sol.

### Incorporation of Thiolated Nanoparticles in Shaped Hydrogel Beads:

Hydrogel beads, containing the above thiolated nanoparticles, were produced by first preparing a mixture of the above thiolated nanoparticle sol (90 mL), distilled water (90 mL), ethoxylated trimethylolpropanetriacrylate (120 g, SR415, Sartomer, Exeter, Pa.), and photoinitiator (1.2 g, IRGACURE 2959, Ciba Specialty Chemicals, Tarrytown, N.Y.). Spherical hydrogel beads of from one to four millimeters in diameter were then prepared from this mixture by photopolymerization, using the method described in Example 1 with reference to FIG. 2 in WO2007/146722 A1. Briefly, the mixture was placed in a funnel, and the mixture exited the funnel through a 2 mm diameter orifice. The exiting mixture fell along the vertical axis of a 0.91 meter long, 51 mm diameter quartz tube extending through a UV exposure zone. This zone included a 240 Watt/inch irradiator with an “H” bulb (25 cm in length) (available from Fusion UV Systems, Gaithersburg, Md.) coupled to an integrated back reflector, such that the length of the bulb was parallel to the vertical axis traveled by the mixture. The mixture exited the exposure zone and quartz tube as spherical hydrogel beads.

### Nitrosylation of the Thiolated Nanoparticles Incorporated in the Hydrogel Beads:

Beads (about 1 gram) prepared above were soaked in 10 mL of a solution of 1.0 gram of sodium nitrite in 100 mM hydrochloric acid for one hour. The beads turned a light red indicating the formation of nitrosothiol groups on the nanoparticles. After these beads were washed with distilled water and placed in 100 mM phosphate buffer at pH 7, they slowly lost their color, indicating the breakdown of the nitrosothiol groups and the formation of nitric oxide.

### Example 14

### Preparation of Saccharide Stabilized Nanoparticles in a Crosslinked Alginate Hydrogel and Generation of Nitric Oxide from the Hydrogel

Thiolated nanoparticle sol, containing nanoparticles with 3-mercaptopropylsilyl and 3-gluconamidopropylsilyl groups (0.9 ml) from Example 12 was placed in a small beaker. This sol, which contained 0.077 mmol of thiol/g of sol was acidified to a pH of 2 by adding 40 μL of 1N HCl, and then an aqueous solution of sodium nitrite (100 μL at 4.78 mg/100 ml was added to nitrosilate the nanoparticles. The reaction is accompanied by the appearance of a red color in the sol. To the resulting sol was added 1 g of a solution of sodium alginate (10 g, MANUCOL LF, available from ISP, Wayne, N.J.) in deionized water 242 g). The resulting mixture was stirred, and then a disposable pipette was used to drop this mixture (one drop at a time) in a vial containing an aqueous solution of calcium chloride (CaCl₂, 10 mg/ml). The resulting hydrogel beads, which were opaque and pink, were allowed to harden for 40 minutes.

The hydrogel beads were tested for NO release as follows. A known amount of hydrogel beads were dispensed in wells of a 12-well petri plate:
Well 1-14 beads (0.53 g hydrogel beads)
Well 2-7 beads (0.23 g hydrogel beads)
Well 3-5 beads (0.1 g hydrogel beads)
   Phosphate-buffered saline solution (3 mL, PBS 10 mM, Sigma, St. Louis, Mo., Cat. No. P3813) was added to each well, and the plate was placed in an incubator at 37° C. At 21.5 hours, a 1 ml sample was collected from each well and frozen at −20° C. until analysis. The remainder of the fluid in each well was removed and a fresh 3 ml of PBS buffer was added to the wells. A second sample was collected from each well at a total cumulative time of 66 hours and frozen at −20° C. until analysis.

The collected samples were analyzed for total nitrite plus nitrate using a commercially available colorimetric kit from Cayman Chemical (Ann Arbor, Mich., Cat. No. 780001). Nitric oxide itself has a short half-life, and the final products of NO degradation are nitrite (NO₂⁻) and nitrate (NO₃⁻). The relative proportion of NO₂⁻ and NO₃⁻ is variable and cannot be predicted with certainty. Thus, the best index of total nitric oxide production is the sum of both, termed NOx. The results are shown in Table 8 below.

### Example 15

### Preparation of Poly(ethylene glycol) Stabilized Nanoparticles in a Crosslinked Alginate Hydrogel and Generation of Nitric Oxide from the Hydrogel

Poly(ethylene glycol) stabilized thiolated nanoparticle sol (1 mL) from Example 13 was placed in a small beaker. The sol was acidified to a pH of 2 with 120 μL of 1 N HCl, and 198 μL of an aqueous solution containing 1.5 weight percent sodium nitrite was added to the sol. The reaction was accompanied by the appearance of a red color in the sol. A 100 mM sodium acetate solution (200 μL) was added to the sol as a buffer, and the resulting mixture was well stirred. A 1 weight percent aqueous solution (140 μL) of sodium carbonate was then added to the mixture, followed by the addition of sodium alginate (1 g) as in Example 14. The resulting mixture was stirred and then a disposable pipette was used to drop this mixture (one drop at a time) in a vial containing an aqueous solution of calcium chloride (10 mg/ml) while stirring with a stir bar. The resulting hydrogel beads, which were translucent and pink, were allowed to harden for 10 minutes.

A known amount of hydrogel beads were dispensed in wells of a 12-well petri plate to be tested for NO release:
Well 1-14 beads (0.53 g hydrogel beads)
Well 2-7 beads (0.23 g hydrogel beads)
Well 3-5 beads (0.1 g hydrogel beads)
   The hydrogel beads were tested for NO release as in Example 14, and the results are shown in Table 8.

TABLE 8
Nitric Oxide Released from Crosslinked Alginate Hydrogels
Containing Saccharide (Example 14) and Poly(ethylene
glycol) (Example 15) Stabilized Nanoparticles

Amount of NOx (micromoles/3 mL)

| Sample | At 21.5 h | At 66 h (cumulative) |
|---|---|---|
| Example 14, Well 1 | 0.39 | 0.66 |
| Example 14, Well 2 | 0.39 | 0.51 |
| Example 14, Well 3 | 0.45 | 0.57 |
| Example 15, Well 1 | 0.16 | 0.28 |
| Example 15, Well 2 | 0.10 | 0.17 |
| Example 15, Well 3 | 0.08 | 0.12 |

### Example 16

### Preparation of Poly(ethylene glycol) Stabilized Nanoparticles in a Crosslinked Hydrophilic (Meth)Acrylate Hydrogel with Lactic Acid and Generation of Nitric Oxide from the Hydrogel

Sol B (25 mL), prepared essentially as described in Example 1, was placed in a beaker, and sodium nitrite (32 mg) was added to the sol. The resulting mixture was stirred, and 870 μL of a 1:10 dilution of lactic acid (PURAC 88% Hi Pure lactic acid, Batch AR9001D, PURAC America, Lincolnshire, Ill.) was added, followed by addition of 1.08 mL of 1N hydrochloric acid to reach a pH of 2.

A hydrophilic (meth)acrylic syrup (15.63 g), consisting of 55-70 parts M-PEG, 10-15 parts MAA-PEG, 30-40 parts HEMA, and 10-20 parts HEA, was placed in a 1 L round bottom flask. The above acidified sol was added to the syrup, and the flask was placed on a rotary evaporator until liquids stopped condensing (about 12 minutes). EDMAB (111.4 mg) and CPQ (111.6 mg) were added to the resulting sol and mixed in by stirring.

The resulting photopolymerizable syrup (about 2 g) was poured into 1 mm deep by 4.1 cm diameter molds and covered with a clear, transparent liner, spreading the syrup evenly within the molds. The syrup was cured under a UV lamp (Black Ray long wavelength UV lamp, Model B-100) through a 455 nm cut-off filter for 15 minutes. The resulting cured hydrogel discs were removed from the molds and the liner and stored in a dessicator covered with foil to keep the hydrogel discs in a dry, dark environment.

Portions of the hydrogel discs were removed using a 1.27 cm diameter hollow punch and placed in wells of 6-well petri plates to be tested for NO release. The small 1.27 cm diameter discs placed in the wells were about 1 mm thick. Ten milliliters of phosphate-buffered saline solution were added to each well. The plate was placed in an incubator at 37° C. At 25.5, 48, 72, and 163 hours, a 1 mL sample was collected from each well. These samples were frozen at −20° C. until analysis. At each sampling, the remainder of the fluid in the well was removed, and a fresh 9 mL of PBS buffer was added to the well. The reason to add back 9 mL (and not 10) is that the small hydrogel discs absorb approximately 1 mL of fluid. This is absorbed after less than 2 hours of incubation and the absorbed volume remains unchanged after that.

The samples were analyzed for total nitrite plus nitrate as in Example 14. The results are shown in Table 9.

### Example 17

### Preparation of Poly(ethylene glycol) Stabilized Nanoparticles in a Crosslinked Hydrophilic (Meth)Acrylate Hydrogel with Hydrochloric Acid and Generation of Nitric Oxide from the Hydrogel

Sol B (25 mL), prepared essentially as described in Example 1, was placed in a beaker, and 600 μL of 1N hydrochloric acid was added, bringing the pH to 2. Sodium nitrite (32 mg) was added to the sol, and the mixture was stirred.

The hydrophilic (meth)acrylic syrup (15.28 g), described in Example 16, was placed in a 1 L round bottom flask. The above acidified sol was added to the syrup, and the flask was placed on a rotary evaporator until liquids stopped condensing (about 12 minutes). EDMAB (111.5 mg) and CPQ (112.0 mg) were added to the resulting sol and mixed in by stirring.

The resulting photopolymerizable syrup was made into cured discs (about 1 mm thick by about 4.1 cm diameter), and the discs stored in a foil covered dessicator as in Example 16. Portions of these discs were removed and tested for NO release by measuring total nitrite plus nitrate as in Example 16. The results are shown in Table 9.

### Example 18

### Preparation of Poly(ethylene glycol) Stabilized Nanoparticles with Thiol-Containing Groups in a Crosslinked Hydrophilic (Meth)Acrylate Hydrogel with Hydrochloric Acid

Sol B (25 mL), prepared essentially as described in Example 1, was placed in a beaker, and 600 μL of 1N hydrochloric acid was added, bringing the pH to 2. Sodium nitrite (32 mg) was added to the sol, and the mixture was stirred.

The hydrophilic (meth)acrylic syrup (15.45 g), described in Example 16, was placed in a 1 L round bottom flask. The above acidified sol was added to the syrup, and the flask was placed on a rotary evaporator until liquids stopped condensing (about 12 minutes). EDMAB (110.4 mg) and CPQ (112.6 mg) were added to the resulting sol and mixed in by stirring.

The resulting photopolymerizable syrup was made into cured discs (about 1 mm thick by about 4.1 cm diameter), and the discs stored in a foil covered dessicator as in Example 16. Portions of these discs were removed and tested for total nitrite plus nitrate as in Example 16. The results are shown in Table 9.

TABLE 9
Total Nitrite and Nitrate Found in Hydrogels
After Incubation in PBS Buffer

| | Cumulative NOx (micromol in 10 mL) | | | |
|---|---|---|---|---|
| Example | At 25.5 hours | At 48 hours | At 72 hours | At 163 hours |
| 16 | 0.052 | 0.068 | 0.081 | 0.099 |
| 17 | 0.025 | 0.035 | 0.045 | 0.061 |
| 18 | 0.01* | 0.020 | 0.028 | 0.043 |

*0.01 micromol per 10 mL is the detection limit for this assay.

It is believed that in Examples 16 and 17 some of the nitrosothiol groups were activated during the UV curing step, thereby causing the cumulative NOx measured above to be lower if cured without exposure to light.

### Example 19

### Prepolymer A:

Benzoyl chloride (0.58 g) is blended at room temperature under an inert atmosphere with 1738 g (1 equivalent) of an approximately 5000 M.W. polyether triol (a copolymer of ethylene oxide and propylene oxide having atactic distribution). Thereafter, 191.4 g (2.2 equivalents) of an 80:20 mixture of 2,4 tolylene diisocyanate:2,6 tolylene diisocyanate is rapidly added to the resultant mixture with aggressive agitation, producing a moderate exothermic reaction. This was maintained at 80-85° C. until the reaction is complete. The progress of the reaction is followed by titrating samples of the mixture for % NCO until the reaction is complete, whereupon the reaction is allowed to cool to room temperature. The upper portion of the reaction mixture is decanted to leave 100% solids prepolymer, designated “Prepolymer A”. This is sealed in a moisture proof glass container.

### Prepolymer B:

Benzoyl chloride (0.415 g) is added with thorough mixing at room temperature under an inert atmosphere to 1738 g (1 equivalent) of the polyether triol as described in Prepolymer A. Thereafter, 337.5 g (2.5 equivalents) of a polymeric MDI polyisocyanate formerly sold under the trade designation “Mondur” 432 is added to the resultant mixture with constant agitation producing an exotherm. This mixture is maintained at 80-85° C. until the reaction is complete as determined by titration for % NCO. The reactants are permitted to cool to 40° C. or less to produce a 100% solids prepolymer, designated “Prepolymer B”. This is sealed in a moisture proof glass container.

### Prepolymer C:

A 4000 M.W. polyoxyethylene glycol (2000 g, 1 equivalent) is reacted with 1814 g (2.2 equivalents) of a 80:20 mixture of 2,4:2,6 tolylene diisocyanate, causing a slight exotherm which was maintained at 70-75° C. until the reaction is complete. This is determined by titration for % NCO. After cooling to room temperature, the prepolymer reaction product is collected and sealed in a glass container, designated “Prepolymer C”.

A 10 g sample of the stable dispersion of nitrosothiolated nanoparticles in water prepared according to Example 1 (nitrosylated sol C) is mixed with 4 g samples of Prepolymers A, B, and C. The contents are stirred vigorously and immediately poured into a mold or coated onto a substrate. Within minutes a crosslinked hydrogel is formed due to reaction of some of the terminal isocyanate groups with water to produce an amine and carbon dioxide and subsequent reaction of the amines with remaining isocyanate groups to form urea linkages.

### Example 20

A 4000 M.W. poly(ethylene glycol) (PEG) 20.0 g. (0.01 equivalent) is mixed with 20 g of the aqueous dispersion of nitrosolated nanoparticles produced in Example 1 (nitrosylated sol C), which is approximately 6.9% SiO₂. This dispersion is stable. The water is removed on a rotary evaporator to produce a 6.9% dispersion of SiO₂ nitrosothiolated poly(ethylene glycol) stabilized nanoparticles in PEG. The PEG dispersion is reacted with 1.43 g (0.01 equivalent) of Isonate 2143 L (a modified MDI having an equivalent weight of 143 g/equivalent available from Dow Chemical. Midland, Mich.). This is mixed well, poured into a Teflon mold and cured at 70-75° C. until the reaction is complete to form a polyurethane hydrogel.

### Example 21

A 3000 M.W. glycerin initiated polyoxyethylene glycol triol 15.0 g. (0.01 equivalent) is mixed with 15 g of the aqueous dispersion of nitrosolated nanoparticles produced in Example 1 (nitrosylated sol C), which is approximately 6.9% SiO₂. This dispersion is stable. The water is removed on a rotary evaporator to produce a 6.9% dispersion of SiO₂ nitrosothiolated poly(ethylene glycol) stabilized nanoparticles in the glycerin initiated polyoxyethylene glycol triol. This dispersion is reacted with 1.43 g (0.01 equivalent) of Isonate 2143 L (a modified MDI having an equivalent with of 143 g/equivalent available from Dow Chemical). This is mixed well, poured into a Teflon mold and cured at 70-75° C. until the reaction is complete to form a polyurethane hydrogel.

### Example 22

### Preparation of Poly(ethylene glycol) Stabilized Nanoparticles in a Crosslinked Guar Gum Hydrogel

A thiolated nanoparticle sol (non-nitrosylated) was prepared as described above in Example 1 (sol B). The volume of the resulting mixture was reduced by rapid vacuum distillation at 25° C., using a Buchi R-205 ROTAVAPOR, to provide a poly(ethylene glycol) stabilized thiolated nanoparticle sol at 39% wt solids. A 2 g aliquot of this solution was nitrosylated with an excess of sodium nitrite following the nitrosylation procedure described in Example 1 (sol C).

To produce a crosslinked guar gum hydrogel, 0.04 g of Guar Gum was added into 0.3 g of propylene glycol (Aldrich Chemical Co.) and the resulting solution was mixed into 1.66 g of the above nitrosylated nanoparticle sol. The crosslinked hydrogel was formed by adding 0.1 g of a 17% wt potassium tetraborate solution.

### Examples 23 and 24

### Preparation of Solid Amorphous Silica Nanoparticles with Polyether and Nitrosothiol Groups

Preparation of Polyether silane I (PES-I):

3-(Triethoxysilyl)propyl isocyanate (Sigma-Aldrich) (5.02 g) was slowly added to a solution of polyetheramine (JEFFAMINE M-1000, Huntsman) (20.32 g) in 100 g dichloromethane (EM Sciences), and the resulting mixture was stirred for 16 hours. The solvent was removed under reduced pressure, and the resulting polyetherurea silane (MW=1247) was isolated as an off-white waxy solid and used without purification.

Preparation of Polyether silane II (PES-II):

3-(Triethoxysilyl)propyl isocyanate (Sigma-Aldrich) (2.48 g) was slowly added to a solution of polyetheramine (JEFFAMINE M-2070, Huntsman) (21.75 g) in 100 g dichloromethane (EM Sciences), and the resulting mixture was stirred for 1 hour. The solvent was removed under reduced pressure, and the resulting polyetherurea silane (MW=2317) was isolated as a yellow-tinged liquid and used without purification.

### Preparation of Thiolated and Polyether Stabilized Nanoparticles:

NALCO 2326 sol (17% wt, Nalco, Naperville, Ill.) (200 g) was combined with 200 g ethanol in a 500 mL, 3-necked round bottom flask fitted with a glass stopper, condenser, and a thermometer. The sol was continuously stirred using a magnetic stirrer. Using a heating mantle, the sol temperature was increased to 80° C. (+/−5° C.) and combined with 3.04 g of 3-mercaptopropyltrimethoxysilane (Alfa Aesar, Ward Hill, Mass.). The sol was maintained at temperature for 8 hours. After 8 hours, a 15 g aliquot was removed to a vial and a Polyether silane (3.00 g of PES-I or 5.38 g of PES-II) prepared above was mixed into the sol. The vial was placed in an oil bath at 70° C. (+/−5° C.) for an additional 8 hours to provide PES-I and PES-II polyether stabilized, thiolated nanoparticle sols.

### Nitrosylation of Polyether Stabilized, Thiolated Nanoparticles:

Each of the above polyether stabilized, thiolated nanoparticle sols (2 g) was placed in a vial, and 1N hydrochloric acid was added to adjust the pH of the sol to 3. Then 0.01 g of sodium nitrite (Aldrich, Milwaukee, Wis.) was added to the sol and vortexed to mix. The resulting nitrosylated sol was red, indicating conversion of the mercapto groups to nitrosothiol groups. The ratio of nitrite to thiol(sulfhydryl) groups on the resulting nanoparticles was 2:1.

Release of NO from Nitrosylated Polyether Stabilized Nanoparticle Sols:

The absorbance spectra of the nitrosylated sols prepared above were measured using a Perkin Elmer Lambda 35 spectrophotometer (Perkin Elmer, Waltham, Mass.). The absorbance of the sol was measured serially over 16 days at 550 nanometers, the visible absorbance peak wavelength of the nitrosothiol groups. Over this time period, the sol was exposed to ambient fluorescent lighting at a temperature of 21° C. Each of the absorbance values was normalized with respect to the initial absorbance. The results shown in Table 10 below indicate that NO was released over the 16 day time period as seen by the decay of the nitrosothiol group absorbance, and that at day 16, the sol maintained approximately 60 percent of the absorbance due to the remaining nitrosothiol groups in the case of the PES-I polyether stabilized nanoparticles (Example 23) and approximately 40% in the case of PES-II polyether stabilized nanoparticles (Example 24).

TABLE 10
Normalized Nitrosothiol Group Absorbance from 0 to 16
Days Under Fluorescent Lighting and Percent NO Released

| | Example 23 | | Example 24 | |
|---|---|---|---|---|
| Time | Normalized | Percent NO | Normalized | Percent NO |
| (Days) | Absorbance¹ | Released² | Absorbance¹ | Released² |
| 0 | 1.00 | 0% | 1.00 | 0% |
| 2 | 0.67 | 33% | 0.63 | 37% |
| 3 | 0.71 | 29% | 0.62 | 38% |
| 4 | 0.72 | 28% | 0.61 | 39% |
| 9 | 0.64 | 36% | 0.52 | 48% |
| 10 | 0.68 | 32% | 0.44 | 56% |
| 13 | 0.59 | 41% | 0.42 | 58% |
| 16 | 0.67 | 33% | 0.39 | 61% |

¹Aₙ/A₀ = Normalized Absorbance
²(A₀ − Aₙ)/A₀ × 100 = Percent NO released as a function of decrease in absorbance at 550 nm.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A composition for releasing nitric oxide, the composition comprising nanoparticles having an exterior surface comprising solid amorphous silica, wherein nitrosothiol-containing groups are attached to the surface, and wherein the nanoparticles are dispersible in an aqueous system.

2. The composition of claim 1, wherein the nanoparticles are solid amorphous silica nanoparticles.

3-4. (canceled)

5. The composition of claim 1, wherein the nanoparticles further comprise stabilizing groups attached to the surface of the nanoparticles, and wherein the stabilizing groups comprise hydrophilic groups selected from the group consisting of —OCH₂CH₂—, —COOH, a salt of —COOH, —CH(OH)CH₂OH, —CH(OH)CH(OH)—, —SO₃H, a salt of —SO₃H, a quaternary amino group, a monosaccharide group, an oligosaccharide group, a plurality of any of the preceding groups, and a combination thereof.

6-11. (canceled)

12. The composition of claim 1, wherein the nanoparticles are dispersed in an aqueous system, which is a combination of water and at least one water dispersible compound.

13-14. (canceled)

15. The composition of claim 1, wherein the composition further comprises a hydrogel; wherein the nanoparticles are distributed in the hydrogel.

16-20. (canceled)

21. A medical device comprising a surface and the composition for releasing nitric oxide of claim 1 any adjacent the surface.

22-24. (canceled)

25. A product comprising a medical device according to claim 21 and a package impervious to water, water vapor, ultraviolet light, and visible light, wherein the composition for releasing nitric oxide is enveloped by the package.

26. A method of treating a subject with nitric oxide, the method comprising:
providing a composition for releasing nitric oxide of claim 1;
contacting the subject with the composition or the medical device; and
releasing nitric oxide at a location where the composition or the medical device contacts the subject.

27. The method of claim 26, further comprising activating the release of the nitric oxide from the composition.

28-29. (canceled)

30. A kit comprising a composition for releasing nitric oxide of claim 1 and an activating agent.

31. A method of making a composition for releasing nitric oxide, the method comprising:
providing solid amorphous silica nanoparticles;
bonding thiol-containing groups to an exterior surface of the solid amorphous silica nanoparticles;
bonding stabilizing groups comprising hydrophilic groups to the exterior surface of the solid amorphous silica nanoparticles; and
nitrosylating the thiol-containing groups to form nitrosothiol-containing groups.

32-33. (canceled)

34. The method of claim 31, wherein bonding the thiol-containing groups to an exterior surface of the solid amorphous silica nanoparticles is carried out by reacting the alkoxysilyl portion of a compound, which includes alkoxysilyl and thiol groups, with the silica nanoparticles.

35-36. (canceled)

37. The method of claim 31, further comprising dispersing the solid amorphous silica nanoparticles, having an exterior surface wherein nitrosothiol-containing groups are attached to the surface, in an aqueous system.

38. The method of claim 31, further comprising distributing the solid amorphous silica nanoparticles, having an exterior surface wherein nitrosothiol-containing groups are attached to the surface, in a hydrogel.

39. A composition for releasing nitric oxide, the composition comprising:
a first part comprising nanoparticles having an exterior surface comprising solid amorphous silica, wherein thiol-containing groups are attached to the surface, and wherein the nanoparticles are dispersible in an aqueous system; and
a second part comprising a nitrite source.

40-44. (canceled)

45. The composition of claim 39, wherein the nanoparticles further comprise stabilizing groups attached to the surface of the nanoparticles, and wherein the stabilizing groups comprise hydrophilic groups selected from the group consisting of —OCH₂CH₂—, —COOH, a salt of —COOH, —CH(OH)CH₂OH, —CH(OH)CH(OH)—, —SO₃H, a salt of —SO₃H, a quaternary amino group, a monosaccharide group, an oligosaccharide group, a plurality of any of the preceding groups, and a combination thereof.

46. The composition of claim 45, wherein the stabilizing groups have a chain length of at least about 10 in-chain atoms, wherein atoms of the hydrophilic groups, other than hydrogen, are included.

47-59. (canceled)

60. A medical device comprising a surface and the composition for releasing nitric oxide of claim 39 adjacent the surface.

61-64. (canceled)

65. A method of treating a subject with nitric oxide, the method comprising:
providing a composition for releasing nitric oxide of claim 39;
combining the first part of the composition with the second part of the composition to provide a composition, wherein nitrosothiol-containing groups are attached to the surface of the nanoparticles;
contacting the subject with the composition for releasing nitric oxide or with the medical device; and
releasing nitric oxide at a location where the composition or the medical device contacts the subject.

66. The method of claim 65, further comprising activating the release of the nitric oxide from the composition.
